(19) United States
(12) Patent Application Publication
(10) Pub. No.: US 2004/0087935 A1
(43) Pub. Date: May 6, 2004

(54) Electrophysiological probes having tissue insulation and /or heating device cooling apparatus

(75) Inventors: Miriam H. Taimisto, San Jose, CA (US); Katie Krueger, San Jose, CA (US); David F. Dueiri, Campbell, CA (US); Robert R. Burnside, Mountain View, CA (US); Russell B. Thompson, Los Altos, CA (US); Steven Yee, Sunnyvale, CA (US)

Correspondence Address: Henricks, Slavin & Holmes LLP, 840 Apollo Street, Suite 200, El Segundo, CA, 90245, US

(73) Assignee: Scimed Life Systems, Inc.

(21) Appl. No.: 10/286,539

(22) Filed: Nov. 1, 2002

(51) Int. Cl. A61B 18/18

(52) U.S. Cl. 606/41

(57) ABSTRACT

Electrophysiological probes having tissue insulation and/or tissue heating device cooling apparatus are disclosed.

### BACKGROUND OF THE INVENTIONS

1. Field of Inventions

The present inventions relate generally to medical devices that support one or more therapeutic elements in contact with body tissue.

2. Description of the Related Art

There are many instances where diagnostic and therapeutic elements must be inserted into the body. One instance involves the treatment of cardiac conditions such as atrial fibrillation, atrial flutter and ventricular tachycardia, which lead to an unpleasant, irregular heart beat, called arrhythmia. Atrial fibrillation, flutter and ventricular tachycardia occur when anatomical obstacles in the heart disrupt the normally uniform propagation of electrical impulses in the atria. These anatomical obstacles (called “conduction blocks”) can cause the electrical impulse to degenerate into several circular wavelets that circulate about the obstacles. These wavelets, called “reentry circuits,” disrupt the normally uniform activation of the chambers within the heart.

A variety of minimally invasive electrophysiological procedures, employing both catheters and surgical probes, have been developed to treat conditions within the body. With respect to the heart, minimally invasive electrophysiological procedures have been developed to treat atrial fibrillation, atrial flutter and ventricular tachycardia by forming therapeutic lesions in heart tissue. The formation of lesions by soft tissue coagulation (also referred to as “ablation”) can provide the same therapeutic benefits provided by surgical procedures, but without invasive, open heart surgery. Atrial fibrillation has, for example, been treated by the formation of one or more long, thin lesions in heart tissue. The treatment of atrial flutter and ventricular tachycardia, on the other hand, requires the formation of relatively large lesions in heart tissue. The inventors herein have determined that conventional methods and apparatus for forming relatively large lesions are susceptible to improvement. More specifically, the inventors herein have determined that the creation of large lesions via conventional methods and apparatus involves the risk of tissue charring and sub-surface gas explosions (or “popping”).

### SUMMARY OF THE INVENTIONS

An apparatus in accordance with one embodiment of a present invention includes a tissue heating device and an insulation device associated with the tissue heating device. Such an apparatus provides a number of advantages over conventional apparatus. For example, the insulation device will prevent tissue adjacent to, but not in contact with, the tissue heating device from being cooled by air, blood or other biological fluids (depending on the procedure). This results in larger lesions than those which would be formed by an otherwise identical tissue heating device in otherwise identical conditions and more accurate tissue temperature sensing.

An apparatus in accordance with one embodiment of a present invention includes a tissue contact surface, a cooling surface and a substantially electrically insulating, substantially thermally conductive layer coextensive with at least a portion of the cooling surface. Such an apparatus, which may be in the form of an electrode or tissue heating device, provides a number of advantages over conventional apparatus. For example, the cooling surface reduces the temperature of the entire apparatus which, in turn, allows wider and deeper lesions to be formed without tissue charring or popping. Additionally, the electrical insulation focuses energy, thereby providing a more efficient apparatus.

An apparatus in accordance with one embodiment of a present invention includes an elongate member including a side wall opening and an expandable tissue cover carried by the elongate member. Such an apparatus, which may be used in conjunction with a tissue heating device, provides a number of advantages over conventional apparatus. For example, tissue cover may be used to prevent tissue adjacent to, but not in contact with, the tissue heating device from being cooled by air, blood or other biological fluids (depending on the procedure) when the tissue heating device is transmitting energy to tissue though the side wall opening. This results in larger lesions than those which would be formed by an otherwise identical tissue heating device in otherwise identical conditions and more accurate tissue temperature sensing.

The above described and many other features and attendant advantages of the present inventions will become apparent as the inventions become better understood by reference to the following detailed description when considered in conjunction with the accompanying drawings.

### BRIEF DESCRIPTION OF THE DRAWINGS

Detailed description of preferred embodiments of the inventions will be made with reference to the accompanying drawings.

FIG. 1A is a plan view of a probe in accordance with a preferred embodiment of a present invention.

FIG. 1B is a side, partial section view showing the distal portion of the probe illustrated in FIG. 1A forming a lesion.

FIG. 1C is a side, partial section view showing the distal portion of a conventional probe forming a lesion.

FIG. 1D is a plan view of an insulation device in accordance with a preferred embodiment of a present invention.

FIG. 1E is a side view of a tissue heating device in accordance with a preferred embodiment of a present invention.

FIG. 1F is an enlarged view of a portion of the tissue heating device illustrated in FIG. 1E.

FIG. 1G is a section view taken along line 1G-1G in FIG. 1B.

FIG. 1H is a section view of a portion of a tissue heating device in accordance with a preferred embodiment of a present invention.

FIG. 2A is a plan view of the distal portion of a probe in accordance with a preferred embodiment of a present invention.

FIG. 2B is a side, partial section view showing the probe distal portion illustrated in FIG. 2A forming a lesion.

FIG. 2C is an end, partial section view showing the probe distal portion illustrated in FIG. 2A forming a lesion.

FIG. 2D is a section view taken along line 2D-2D in FIG. 2A.

FIG. 3A is a side, partial section view showing the distal portion of a probe in accordance with a preferred embodiment of a present invention forming a lesion.

FIG. 3B is an end view showing the probe distal portion illustrated in FIG. 3A forming a lesion.

FIG. 4A is a plan view of a probe and sheath in accordance with a preferred embodiment of a present invention.

FIG. 4B is an enlarged view of the distal portion of the probe and sheath illustrated in FIG. 4A.

FIG. 4C is a plan view of an insulation device in accordance with a preferred embodiment of a present invention.

FIG. 5A is a side view of the distal portion of a probe in accordance with a preferred embodiment of a present invention.

FIG. 5B is a section view taken along line 5B-5B in FIG. 5A.

FIG. 6A is a plan view of a probe in accordance with a preferred embodiment of a present invention.

FIG. 6B is a side, partial section view showing the distal portion of the probe illustrated in FIG. 6A forming a lesion.

FIG. 6C is a section view taken along line 6C-6C in FIG. 6B.

FIG. 6D is a side view of the distal portion of a probe in accordance with a preferred embodiment of a present invention.

FIG. 6E is a side view of the distal portion of a probe in accordance with a preferred embodiment of a present invention.

FIG. 6F is a side view of the distal portion of a probe in accordance with a preferred embodiment of a present invention.

FIG. 6G is a side view of the distal portion of a probe in accordance with a preferred embodiment of a present invention.

FIG. 7A is a plan view of the distal portion of an apparatus in accordance with a preferred embodiment of a present invention.

FIG. 7B is a plan view of the proximal portion of the apparatus illustrated in FIG. 7A.

FIG. 7C is an end, partial section view showing the apparatus illustrated in FIG. 7A forming a lesion.

FIG. 7D is a side view of a portion of the apparatus illustrated in FIG. 7A.

FIG. 7E is a section view taken along line 7E-7E in FIG. 7D.

FIG. 8A is a plan view of the distal portion of an apparatus in accordance with a preferred embodiment of a present invention.

FIG. 8B is a plan view of the proximal portion of the apparatus illustrated in FIG. 8A.

FIG. 8C is an end, partial section view showing the apparatus illustrated in FIG. 8A forming a lesion.

FIG. 8D is a side view of a portion of the apparatus illustrated in FIG. 8A.

FIG. 8E is a section view taken along line 8E-8E in FIG. 8D.

FIG. 8F is an end view of the apparatus illustrated in FIG. 8A in an unflexed state.

### DETAILED DESCRIPTION OF THE PREFERRED EMBODIMENTS

The following is a detailed description of the best presently known modes of carrying out the inventions. This description is not to be taken in a limiting sense, but is made merely for the purpose of illustrating the general principles of the inventions.

The detailed description of the preferred embodiments is organized as follows:

I. Introduction

II. Tissue Insulation Structures

III. Tissue Heating Devices With A Cooling Region

IV. Expandable Tissue Covers

V. Temperature Sensing and Power Control

The section titles and overall organization of the present detailed description are for the purpose of convenience only and are not intended to limit the present inventions.

I. Introduction

The present inventions may be used within body lumens, chambers or cavities for diagnostic or therapeutic purposes in those instances where access to interior bodily regions is obtained through, for example, the vascular system or alimentary canal and/or with minimally invasive surgical procedures. For example, the inventions herein have application in the diagnosis and treatment of arrhythmia conditions within the heart. The inventions herein also have application in the diagnosis or treatment of ailments of the gastrointestinal tract, prostrate, brain, gall bladder, uterus, and other regions of the body.

With regard to the treatment of conditions within the heart, the present inventions are designed to produce intimate tissue contact with target substrates associated with various arrhythmias, namely atrial fibrillation, atrial flutter, and ventricular tachycardia. For example, the distal portion of a probe in accordance with a present invention can be used to create lesions to treat atrial fibrillation, atrial flutter and ventricular tachycardia.

The inventions herein are also applicable to both catheter-based probes and surgical probes. Catheter-based probes used to create lesions typically include a relatively long and flexible catheter body that supports a soft tissue coagulation electrode (or other tissue heating device) on its distal end and/or a series of spaced soft tissue coagulation electrodes near the distal end. The portion of the catheter body that is inserted into the patient is typically from 23 to 55 inches in length and there may be another 8 to 15 inches, including a handle having steering controls, outside the patient. The length and flexibility of the catheter body allow the catheter to be inserted into a main vein or artery (typically the femoral vein), directed into the interior of the heart, and then manipulated such that the coagulation electrodes contact the tissue that is to be ablated. Fluoroscopic imaging is used to provide the physician with a visual indication of the location of the catheter.

Surgical probes, on the other hand, are configured such that the distal end of a surgical probe may be placed directly in contact with the targeted tissue area by a physician during a surgical procedure, such as open heart surgery. Here, access may be obtained by way of a thoracotomy, median sternotomy, or thoracostomy. Exemplary surgical probes are disclosed in U.S. Pat. No. 6,142,994, which is incorporated herein by reference. Surgical probes preferably include a handle and a relatively short shaft that supports one or more soft tissue coagulation electrodes (or other tissue heating devices) at or near the distal end. Preferably, the length of the shaft is about 4 inches to about 18 inches. The shaft is also preferably relatively stiff. In other words, the shaft is either rigid, malleable, or somewhat flexible. A rigid shaft cannot be bent. A malleable shaft is a shaft that can be readily bent by the physician to a desired shape, without springing back when released, so that it will remain in that shape during the surgical procedure. Thus, the stiffness of a malleable shaft must be low enough to allow the shaft to be bent, but high enough to resist bending when the forces associated with a surgical procedure are applied to the shaft. A somewhat flexible shaft will bend and spring back when released. However, the force required to bend the shaft must be substantial.

II. Tissue Insulation Structures

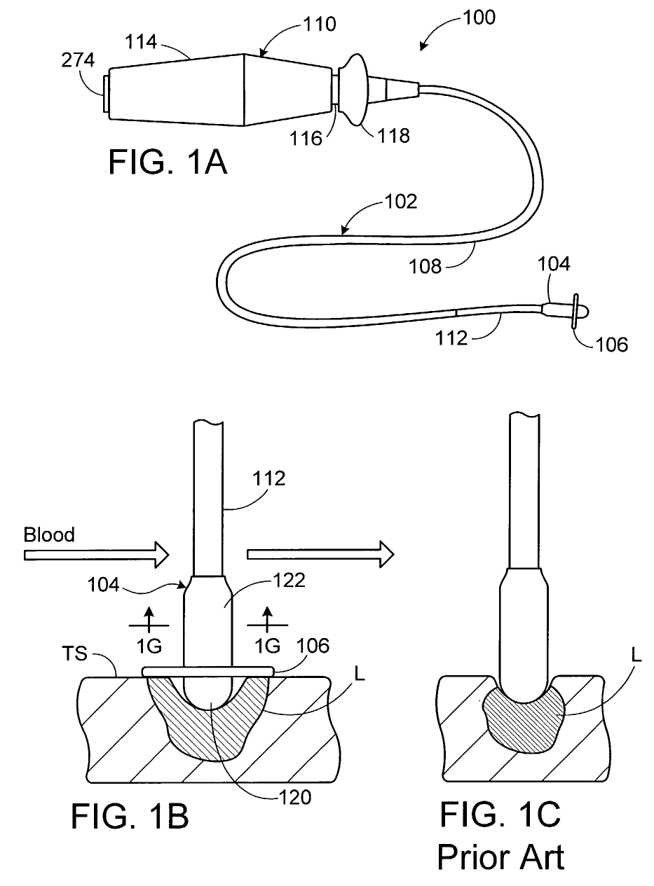

As illustrated for example in FIGS. 1A-1H, a catheter-based probe 100 in accordance with a preferred embodiment of a present invention includes a hollow, flexible catheter body 102, an electrode 104 (or other tissue heating device) and an insulation device 106. The catheter body 102 is preferably steerable and formed from two tubular parts, or members, both of which are non-conductive. The proximal member 108 is relatively long and is attached to a handle 110, while the distal member 112, which is relatively short, carries the electrode 104. The proximal member 108 may be formed from a biocompatible thermoplastic material, such as a Pebax®) material (polyether block amide) and stainless steel braid composite or a polyethylene and stainless steel braid composite, which has good torque transmission properties. An elongate guide coil (not shown) may be provided within the proximal member 108. The distal member 112 may be formed from a softer, more flexible biocompatible thermoplastic material such as unbraided Pebax® material, polyethylene, or polyurethane. The proximal and distal members 108 and 112, which are about 6 French to about 8 French in diameter, may be either bonded together with an overlapping thermal bond or adhesively bonded together end to end over a sleeve in what is referred to as a “butt bond.”

With respect to steering, the exemplary catheter probe 100 may be provided with a conventional steering center support and steering wire arrangement (not shown). The proximal end of the steering center support is mounted near the distal end of the proximal member 108, while the distal end of the steering center support is secured to (but electrically insulated from) the electrode 104. A steering wire is secured to one side of the steering center support and extends through the catheter body 102 to the handle 110, which is also configured for steering. More specifically, the exemplary handle 110 includes a handle body 114 and a piston 116 that is slidable relative to the handle body. A thumb rest 118 is provided on the piston 116. The proximal end of the steering wire is secured to the handle body 114, while the proximal end of the catheter body 102 is secured to the piston 116. Movement of the piston 116 relative to the handle body 114 will cause the catheter body distal member 112 to deflect relative to the proximal member 108. Additional details concerning suitable handles and steering arrangements are disclosed in U.S. Pat. Nos. 6,013,052 and 6,287,301, each of which is incorporated herein by reference. Nevertheless, it should be noted that the present inventions are not limited to steerable probes or any particular type of steering arrangement in those probes which are steerable.

The exemplary electrode 104 is preferably hollow and molded from electrically conducting materials such as silver, platinum, gold, stainless steel or platinum iridium. The diameter of the exemplary electrode 104 will typically range from about 6 French to about 8 French, while the length is typically about 4 mm to about 10 mm. It should be noted, however, that the present inventions are not limited to the use of electrodes and other tissue heating devices, such as laser arrays, ultrasonic transducers, microwave electrodes, and ohmically heated hot wires, may also be employed in embodiments of the present inventions.

Referring to FIG. 1B, the exemplary insulation device 106 is positioned between the electrode distal portion 120 and proximal portion 122 and will cover a portion of the tissue surface TS when the electrode distal portion is urged against the tissue surface. The insulation device 106 prevents air, blood or other biological fluids (depending on the procedure) from cooling the tissue below the insulation device while the electrode is transferring energy (such as RF energy) to heat the tissue. The lesion L that is formed will be generally coextensive with the perimeter of the insulation device 106. Such a lesion L will also be wider than a lesion formed with an essentially identical electrode, but without the insulation device 106, in otherwise identical conditions (i.e. identical body location, power level, etc.). [See FIG. 1C.] The proximal portion 122 of the electrode 104 will be exposed during the lesion formation process and, therefore, will transfer heat to the air, blood or other biological fluid. The heat transfer reduces the temperature of the entire electrode 104 and facilitates the formation of relatively deep lesions without causing charring or popping in the tissue in contact with the distal portion 120 of the electrode. As such, the combination of the insulation device 106 and its positioning relative to the electrode 104, which leaves the proximal portion 122 exposed for cooling, results in lesions with greater volume (i.e. are wider and deeper) than conventional probes without insulation devices. Such an arrangement also improves the accuracy of tissue temperature sensing.

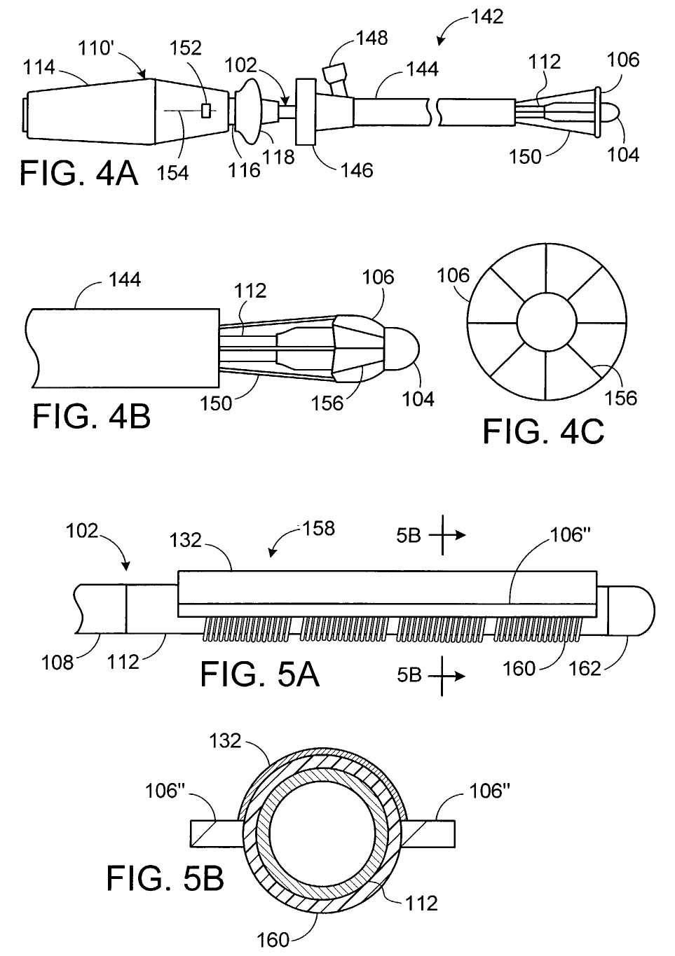

The insulation device shape, size and material may be varied as desired to suit particular situations. Turning to FIG. 1D, the insulation device 106 may, for example, be annularly-shaped and include an inner surface 124 that is positioned against the electrode 104 or other tissue heating device. Other suitable shapes include, but are not limited to, squares, rectangles, ellipses and other geometric shapes. With respect to dimensions, the outer diameter (or width in a non-circular implementation) may range from slightly greater than the diameter of the electrode 106 to about 5 times the diameter of the electrode. The exemplary insulation device 106 is about 4.0 mm to about 5.4 mm, i.e. about two times the diameter of a typically sized 6-8 French electrode. The thickness of the insulation device 106 will depend on the flexibility of the insulating material and the desired level of insulation. With respect to materials, the exemplary insulation device 106 is preferably formed from a relatively flexible, electrically insulating material such as room temperature vulcanized (RTV) silicones and urethanes, Pebax®, siliconized rubber and thermoplastics. The thickness of insulation device formed from these materials would typically be about 0.005 inch to about 0.020 inch, which will result in a level of flexibility that is sufficient to allow the insulation device 106 to deflect when pulled into a sheath (such as the sheath 144 illustrated in FIG. 4A) or other introducer. The thermal conductivity will be relatively low and preferably about 0.1 w/m·k to about 0.3 w/m·k. The insulation device 106 may also be provided with physical structures (such as the creases 156 illustrated in FIG. 4C) that allow the insulation device to more easily deflect when pushed or pulled into an introducer.

The electrode 104 and insulation device 106 are preferably connected to one another in order to prevent the insulation device from separating from the electrode during use. Although the present inventions are not limited to any particular methods or structures for securing the electrode 104 and insulation device 106 to one another, the exemplary electrode is provided with an indentation 126 that receives the insulation device. [Note FIGS. 1E and 1F.] The insulation device 106 is preferably formed around the indentation 126 during an insert molding process. In order to augment the connection, the indentation 126 may be provided with mechanical interlocks, such as holes 128 and/or projections 130, into and around which the insulation material will flow during the molding process.

As illustrated for example in FIG. 1G, the proximal portion 122 of the exemplary electrode 104 is preferably provided with an electrically insulating, thermally conductive coating 132. Such a coating will allow the electrode 104 to dissipate heat by way of the proximal portion 122, while at the same time preventing energy (such as RF energy) from being transmitted into the blood or other biological fluid. This focuses the energy being transmitted by the electrode 104 into the tissue and provides a more efficient device. Suitable electrically insulating, thermally conductive coatings include epoxies, thin-walled heat shrink tubing, and injection molded coatings formed from materials such as polyether block amide (e.g. Konduit® PEA Series by LNP Engineering Plastics, located in Exton, Pa.). The thickness of the electrically insulating, thermally conductive coating 132 would be about 0.0005 inch to about 0.015 inch, while the thermal conductivity of the coating would be relatively high and preferably about 200 w/m·k to about 500 w/m·k.

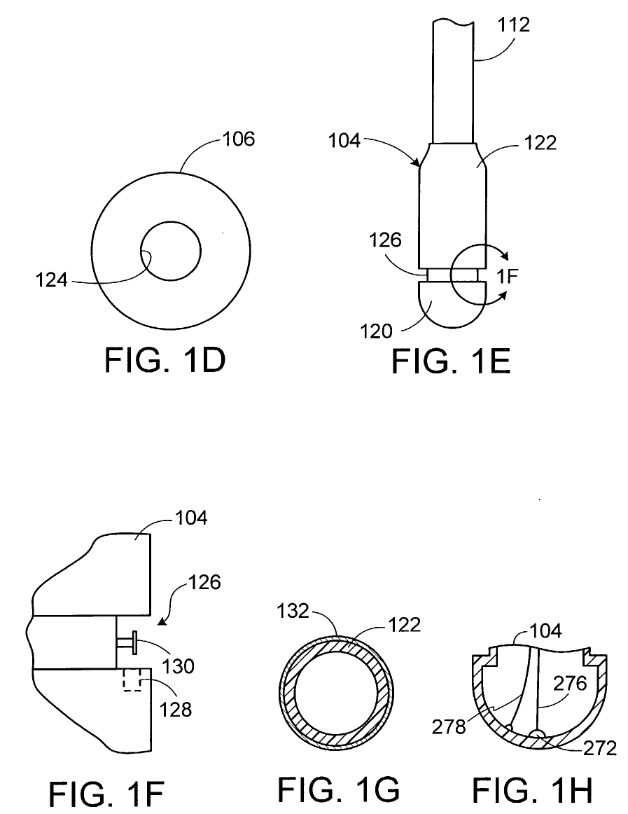

The exemplary insulation device 106 illustrated in FIGS. 1A and 1B extends radially outwardly from the electrode 104 in a plane perpendicular to the longitudinal axis of the electrode. Nevertheless, the present inventions are not limited to such an arrangement. The insulation device 106 may, for example, be slanted relative to the longitudinal axis. Probes in accordance with the present inventions may also include insulation devices that extend longitudinally along the sides of an electrode or other tissue heating device. One example of such a probe is generally represented by reference numeral 134 in FIGS. 2A-2D. The exemplary catheter-based probe 134 is substantially similar to the probe 100 described above with reference to FIGS. 1A-1H and similar elements are represented by similar reference numerals. The probe 134 includes a steerable catheter body 102 with a handle (not shown) at the proximal end and an electrode 104′ or other tissue heating device at the distal end of the distal member 112. Here, however, the insulation device 106′ extends longitudinally along the sides of the electrode 104′ and around the distal end of the electrode. As a result of this configuration, and as illustrated for example in FIGS. 2B and 2C, the electrode 104′ and insulation device 106′ may be positioned on the tissue surface TS such that the longitudinal axis of the electrode is generally parallel to the tissue surface. The electrode 104′ and insulation device 106′ will produce a lesion L that is wider (i.e. larger in a direction perpendicular to the longitudinal axis of the electrode) than would be produced by an otherwise identical electrode without the insulation device. [Note the wider lesion illustrated in FIG. 2C.] The lesion will also be slightly longer. [See FIG. 2B.]

As illustrated for example in FIG. 2D, the electrode bottom portion 136 and top portion 138 are on opposite sides of the exemplary insulation device 106′. The bottom portion 136 is used to transfer energy (such as RF energy) to the tissue. The top portion 138 is exposed to air, blood or other biological fluids (depending on the procedure) and transfers heat to the air, blood or other biological fluid to reduce the temperature of the entire electrode 104′. As noted above, such electrode cooling facilitates the formation of deeper lesions without tissue charring and popping. The top portion 138 may also be provided with the electrically insulating, thermally conductive coating 132 described above with reference to FIG. 1G.

The exemplary electrode 104′ and insulation device 106′ will preferably be secured to one another in the same manner as the electrode 104 and insulation device 106. To that end, the electrode 104′ is provided with an indentation 126′ that is coextensive with the insulation device 106′ (FIG. 2D) and may also be provided with suitable mechanical interlocks such as holes and projections. With respect to size, the insulation device 106′ preferably extends outwardly about 0.05 inch to about 0.20 inch from the exterior surface on each side of the electrode 104′. The insulation device 106′ may also be symmetrical about the electrode 104′ (as shown) or asymmetrical.

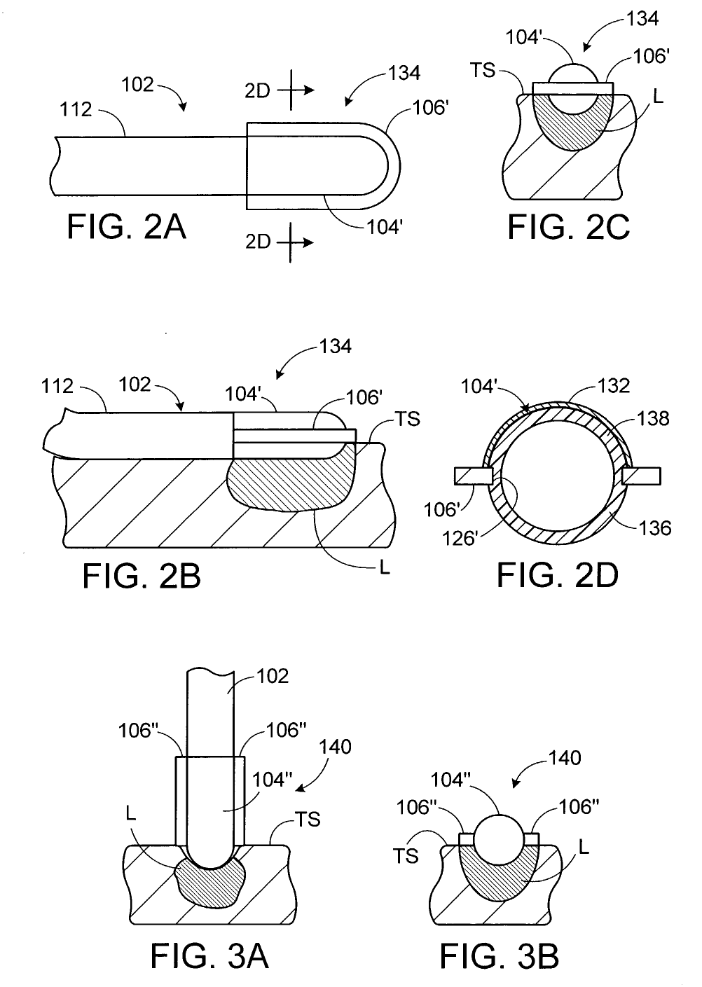

The exemplary embodiment illustrated in FIGS. 2A-2D may also be modified by eliminating the portion of insulation device 106′ that extends around the distal end of the electrode 104′. Referring more specifically to FIGS. 3A and 3B, an exemplary probe 140 (which is otherwise substantially similar to the probe 100 illustrated in FIGS. 1A-1H) includes a pair of insulation devices 106″ that extend along the sides of an electrode 104″. Such an arrangement allows the electrode 104″ and insulation devices 106″ to be used in the manner illustrated in FIG. 3A, with the longitudinal axis of the electrode perpendicular to the tissue surface TS, as well as in the manner illustrated in FIG. 3B, with the longitudinal axis of the electrode parallel to the tissue surface. The top portion of the electrode 104″ may also be provided with an electrically insulating, thermally conductive coating similar to that illustrated in FIG. 2D that is coextensive with the insulation devices 106″.

Probes in accordance with the present inventions may also be provided with structures that facilitate the deflection of the insulation device so that it may be more readily moved into a sheath or other introducer. Two examples of such structures, which may be utilized alone or in combination with one another, are illustrated in FIGS. 4A-4C. Referring first to FIG. 4A, the exemplary probe 142 illustrated therein is substantially similar to the exemplary probe 100 illustrated in FIG. 1A and similar elements are represented by similar reference numbers. The probe 142 in the exemplary illustration has been inserted though a sheath 144, which is provided with a hemostasis valve 146 and a fluid port 148, and the electrode 104 and insulation device 106 have been pushed through the distal end of the sheath. In order to facilitate retraction of the insulation device 106 back into sheath 140, the exemplary probe 142 is provided with a plurality of pull wires 150 that are secured at various points around the outer perimeter of the insulation device. The pull wires 150 enter the catheter body 102 by way of holes (not shown) in the distal member 112 and extend into the handle 110′ where they are connected to a slider 152 that rides along a slot 154. Moving the slider 152 proximally will pull the wires 150 and insulation device 106 from the orientation illustrated in FIG. 4A to the orientation illustrated in FIG. 4B, which allows the insulation device to be more easily retracted into the sheath 144. Alternatively, splines may be used in place of the pull wires 150 in order to allow the perimeter of the insulation device to be pushed distally as well as pulled proximally.

As illustrated for example in FIG. 4C, the exemplary insulation device 106 may be provided with creases 156 or other physical structures (such as score lines) that make it easier for the insulation device to deflect from the orientation illustrated in FIG. 4A to the orientation illustrated in FIG. 4B. The creases 156 or other physical structures may also be incorporated into insulation devices that are not used in conjunction with pull wires.

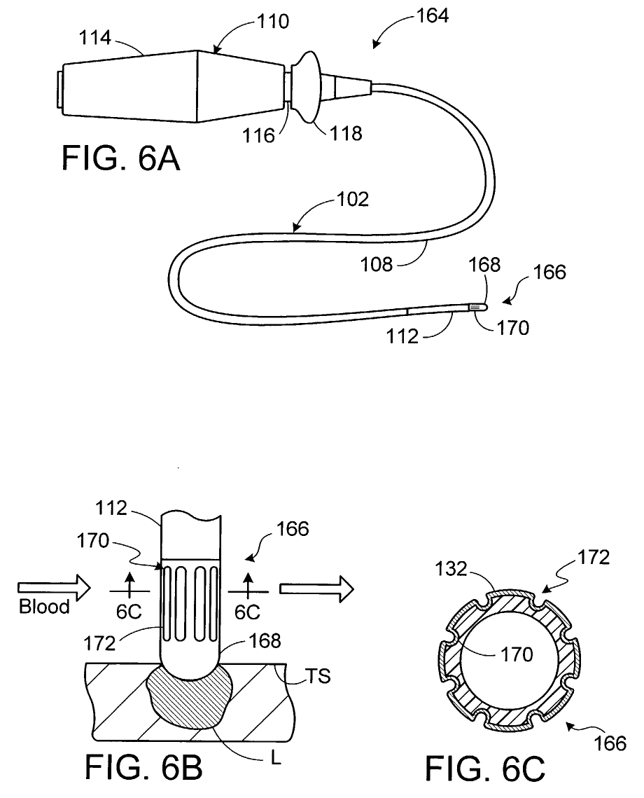

Insulation devices may also be used in conjunction with probes that carry multiple electrodes. One example of such a probe, which is generally represented by reference numeral 158 in FIGS. 5A and 5B, is in many ways similar to the steerable probe 100 illustrated in FIGS. 1A-1H and similar elements are represented by similar reference numerals. Here, however, a plurality of spaced coil electrodes 160 are carried by the catheter body distal member 112. A pair of longitudinally extending thermal insulation devices 106″ and an electrically insulating, thermally conductive coating 132 are also carried by the distal member 112. The thermal insulation devices 106″ may be secured to the electrodes 160 and distal member 112 with suitable adhesives or techniques such as ultrasonic welding. Additionally, in the exemplary probe 158, the inner steering center support is connected to a tip member 162 that is secured to the distal end of the catheter body distal member 112.

It should also be emphasized that, although the exemplary probes illustrated in FIGS. 1A-5B are steerable catheter-based probes, the inventions are not so limited. To that end, the inventions also encompass non-steerable catheter-based probes and surgical probes, both with and without steering capabilities. The insulation devices described above may also be used in conjunction with the electrodes described in Section III. Additionally, although the insulation devices are carried by the electrodes in the exemplary embodiments described above, there may be some instances in which they are carried by the associated catheter or surgical probe shaft just proximal to the electrode, such as when a relatively small electrode is employed.

III. Tissue Heating Devices with a Cooling Region

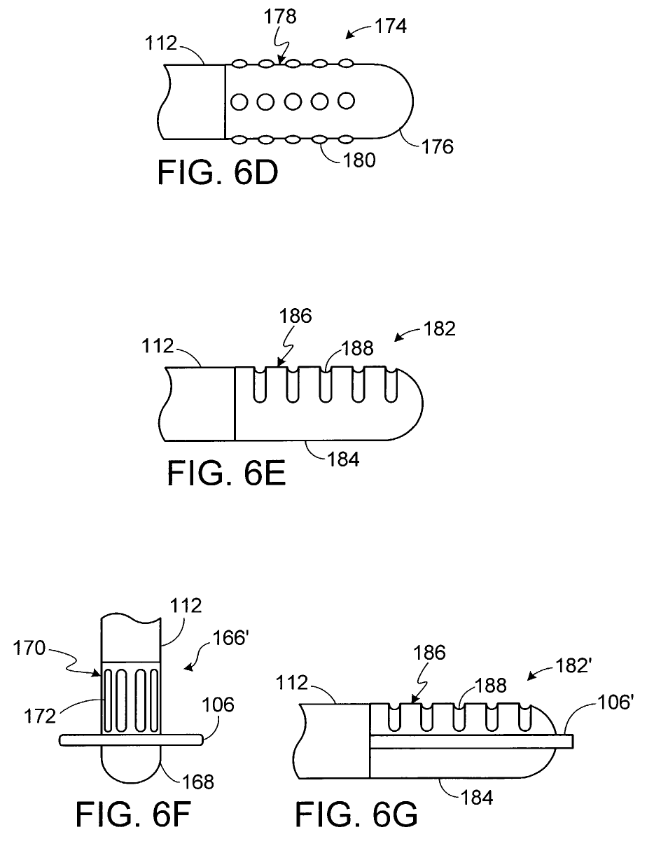

The exemplary steerable catheter-based probe generally represented by reference numeral 164 in FIG. 6A is substantially similar to the probe illustrated in FIG. 1A and similar elements are represented by similar reference numerals. Here, however, the exemplary probe carries an electrode 166 that includes a tissue contact surface 168 and an electrode cooling surface 170. The electrode cooling surface 170 has a plurality of discontinues that increase the surface area of that portion of the electrode, as compared to a continuous surface such as a surface that defines a perfect circle in cross-section. The increase in surface area will correspondingly increase the amount of heat transferred from the electrode cooling surface 170 to air, blood or other biological fluids, thereby reducing the temperature of the entire electrode (including the tissue contact surface). This, in turn, allows wider and deeper lesions to be formed without tissue charring or popping.

The present inventions are not limited to any particular surface discontinuities. In the exemplary embodiment illustrated in FIGS. 6A-6C, the discontinuities are in the form of longitudinally extending indentations 172. The exemplary indentations 172 are generally parallel to the longitudinal axis of the electrode 166 and extend from the proximal end of the tissue contact surface 168 to an area near the intersection with distal end of the catheter body distal member 112. Alternatively, the indentations could be in the form of rings that are generally perpendicular to the longitudinal axis and extend around the electrode cooling surface 170. Another alternative indentation arrangement is a plurality of dimples.

The discontinuities may also be in the form of protuberances that extend outwardly from the otherwise continuous electrode surface. As illustrated for example in FIG. 6D, an electrode 174 in accordance with another preferred embodiment includes a tissue contact surface 176 and an electrode cooling surface 178. A plurality of spaced protuberances 180 are formed on the electrode cooling surface 178 in order to increase the surface area of the cooling surface. Alternatively, the electrode 174 may include a series of protuberances that extend parallel to the longitudinal axis of the electrode (in a manner similar to the indentations 172 in FIGS. 6A-6C) or a series of ring-like protuberances that are perpendicular to the longitudinal axis and extend around the electrode.

The exemplary electrodes illustrated in 6A-6D include an electrode cooling surface that is located proximally of the tissue contact surface. As illustrated for example in FIG. 6E, an electrode 182 in accordance with another preferred embodiment includes a tissue contact surface 184 and an electrode cooling surface 186 that are parallel to one another and to the longitudinal axis of the electrode. This allows the electrode 182 to be positioned with its longitudinal axis parallel to the tissue surface, the tissue contact surface 184 in contact with tissue, and the electrode cooling surface 186 exposed to air, blood or other biological fluids. The electrode cooling surface 186 is also provided with a plurality of discontinuities that increase surface area. More specifically, and although any of the discontinues described above may be employed, the exemplary electrode 182 includes a plurality of semi-circular (i.e. about 180°) indentations 188.

It should also be noted that insulation devices, such as those described above with reference with FIGS. 1A-5C, may be used in conjunction with the exemplary electrodes illustrated in FIGS. 6A-6E. As illustrated for example in FIG. 6F, an exemplary electrode 166′, which is otherwise identical to the electrode 166, is provided with an insulation device 106. Similarly, the exemplary electrode 182′ illustrated in FIG. 6G, which is otherwise identical to the electrode 182, is provided with an insulation device 106′.

The exemplary electrodes described above with reference to FIGS. 6A-6G are preferably formed from conductive materials such as silver, platinum, gold, stainless steel or platinum iridium. The electrodes are also typically hollow, unitary structure with a wall thickness of about 0.015 mm to about 0.030 mm and the indentations are typically about 0.0075 mm to about 0.015 mm deep. The protuberances typically cover up to 90% of the electrode cooling surface.

Regardless of the configuration and position of the electrode cooling surface, and whether or not an insulation device is employed, the electrode cooling surface may be provided with an electrically insulating, thermally conductive coating 132 (see, e.g., the electrode cooling surface 170 illustrated in FIG. 6C). The coating will allow the electrode to dissipate heat by way of the electrode cooling surface, while at the same time preventing energy (such as RF energy) from being transmitted into the blood or other biological fluids. This focuses the energy being transmitted by the electrode into the tissue and provides a more efficient device.

IV. Expandable Tissue Covers

In the exemplary embodiments described above with reference to FIGS. 1A-5B, the devices that cover tissue and insulate it from the cooling effects of air, blood or other biological fluids (depending on the procedure) are carried by the tissue heating probes themselves. Devices that cover tissue may also be carried by separate structures which are used in combination with tissue heating probes.

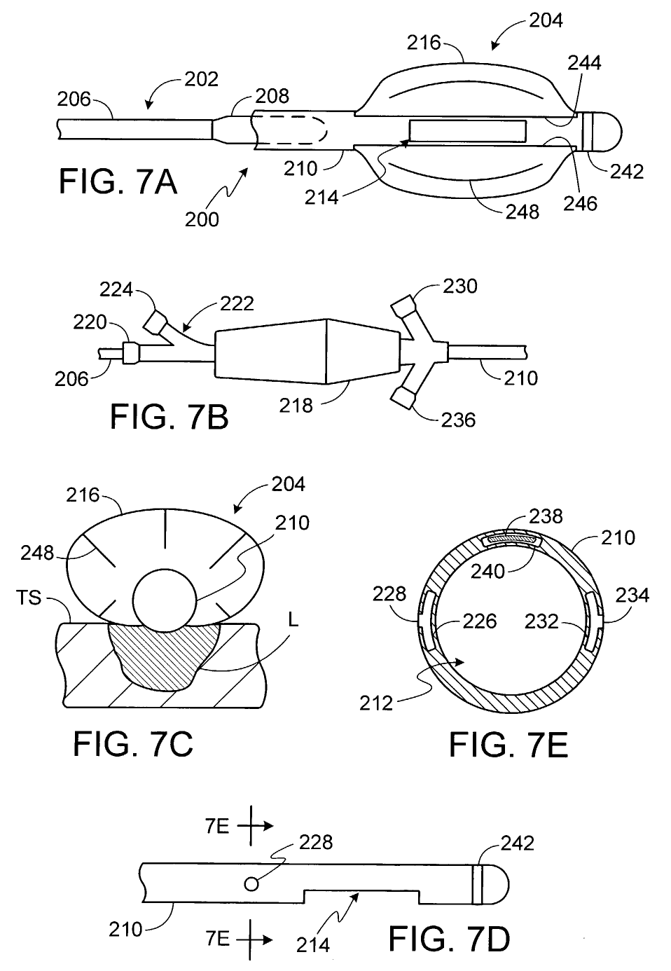

As illustrated for example in FIGS. 7A-7E, an apparatus 200 in accordance with a preferred embodiment of a present invention includes a tissue heating probe 202 and a tissue covering probe 204. The exemplary tissue heating probe 202 includes an elongate catheter body 206 and an electrode 208 or other tissue heating element. The exemplary tissue covering probe 204 includes an elongate tubular body 210, with a central lumen 212 and an opening 214, and an expandable tissue cover 216. The central lumen 212 is configured to slidably receive the tissue heating probe 202 so that the tissue heating probe may be advanced distally until the electrode 208 is aligned with the opening 214 and a portion of the electrode is exposed. Thus, when the tubular body opening 214 is positioned on the tissue surface TS, the electrode 208 may be aligned with the opening 214 and used to transmit lesion forming energy to the tissue.

The proximal end of the tubular body 210 is secured to a handle 218, which is provided with a port 220 through with the tissue heating probe 202 may be inserted. The port 220 is part of a Y-adapter 222, which also includes a hemostasis valve 224. Preferably, the tubular body 210 extends through the interior of the handle 218 to the Y-adapter 222 so that the tissue heating probe 202 is able to pass through the handle.

Referring more specifically to FIGS. 7A and 7C, the tissue cover 216 is preferably sized and positioned such that it will surround the opening 214. The expanded tissue cover 216 will prevent the tissue surrounding the opening 214 from being cooled by air, blood or other biological fluids. As a result, the lesion L that is formed will be generally coextensive with the perimeter of the portion of the cover 216 that is in contact with the tissue. Such a lesion L will also be larger than a lesion formed with an essentially identical electrode, but without the tissue cover 216, in otherwise identical conditions (i.e. body location, power level, etc.). Such an arrangement also improves the accuracy of temperature sensing.

The exemplary expandable tissue cover 216 is an inflatable device that may be inflated with water, saline, or other biocompatible fluids. To that end, and referring to FIGS. 7B, 7D and 7E, the exemplary tubular body 210 is provided with an infusion lumen 226 with an outlet 228 that is located within the tissue cover 216. The infusion lumen 226 also includes an inlet that is connected to a port 230 on the handle 218. With respect to removal of the fluid, the tubular body 210 is provided with a ventilation lumen 232 that has an inlet 234 which is located within the tissue cover 216. The ventilation lumen 232 also includes an outlet that is connected to a port 236 on the handle 218. It should be noted that the exemplary dual infusion/ventilation lumen arrangement may be replaced with a single lumen that is used for both infusion and ventilation.

The exemplary tubular body 210 is preferably formed from a flexible biocompatible material such as polyurethane. Additionally, with respect to dimensions, the outer diameter is preferably about 0.118 inch to about 0.131 inch and the wall thickness is about 0.010 inch to about 0.020 inch. In order to improve tissue contact, the tubular body may also be provided with a stiffening member 238 with a predefined curvature. The stiffening member 238, which is located within a lumen 240, has a predefined curvature over the length of the opening 214 and is positioned 180° from the opening. A radiopaque ring 242 may be positioned near the distal end of the tubular body 210, which is preferably a closed distal end, in order to facilitate fluoroscopic imaging.

Although the respective configurations of the tubular body opening 214 and expandable tissue cover 216 may vary from application to application to suit particular situations, the configurations in the exemplary embodiment have been chosen to insure close contact between the tissue cover and the tissue surface TS. The exemplary opening 214 is about 0.225 inch to about 0.550 inch in length and occupies approximate 25% to 50% of the perimeter of tubular body 210. The tissue cover inner edges 244 and 246 are positioned closely adjacent to the edges of the opening 214. The exemplary tissue cover 216 is also generally elliptical and positioned such that its longitudinal axis is offset from the longitudinal axis of the tubular body 210. [Note FIG. 7C.] Additionally, the tissue cover 216 will initially extend downwardly (in the orientation illustrated in FIG. 7C) from the inner edges 244 and 246. With respect to size, the tissue cover is about 0.457 inch to about 0.792 inch in length, i.e. it extends about 0.15 inch beyond the longitudinal edges of the opening 214, and is about 0.237 inch to about 0.354 inch wide when expanded, i.e. it extends about 0.079 inch to about 0.118 inch from the sides of the tubular body 210.

The exemplary tissue cover 216 is preferably formed from a flexible, thermally insulating material such as polyethylene terephthalate (PET). The tissue cover 216 may also be provided with physical structures, such as creases 248 (FIG. 7C), that allow the tissue cover to more easily fold up for retraction into a sheath.

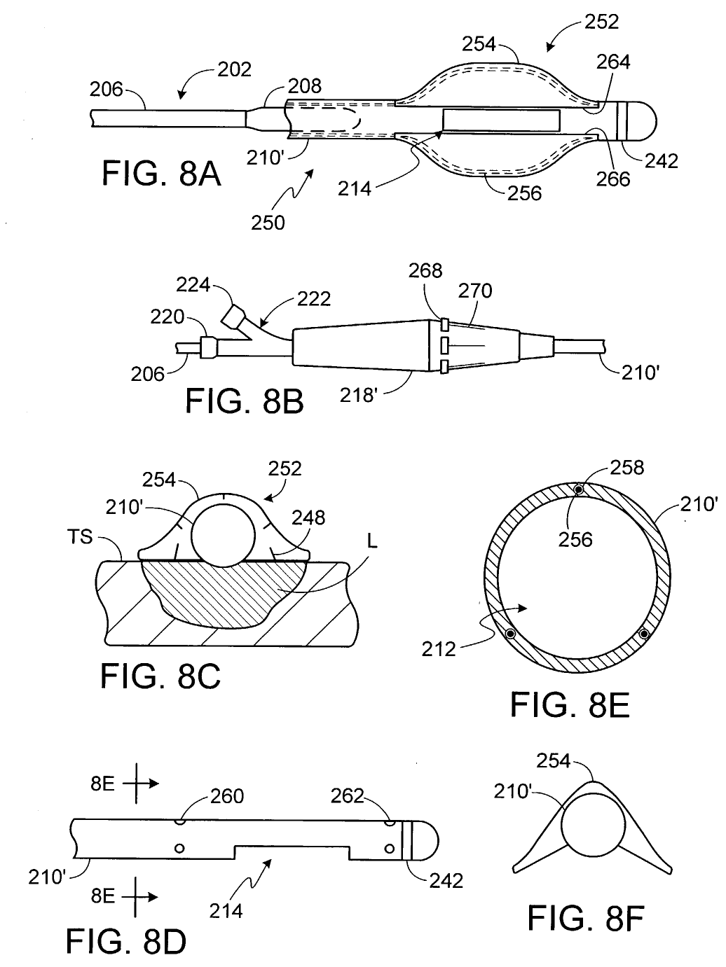

Turning to FIGS. 8A-8F, an apparatus 250 in accordance with a preferred embodiment of a present invention includes the aforementioned tissue heating probe 202 and a tissue covering probe 252. The exemplary tissue covering probe 252 includes an elongate tubular body 210′, with a central lumen 212 and an opening 214, and an expandable tissue cover 254. The central lumen 212 is configured to slidably receive the tissue heating probe 202 so that the tissue heating probe may be advanced distally until the electrode 208 is aligned with the opening 214 and a portion of the electrode is exposed. Thus, when the tubular body opening 214 is positioned on the tissue surface TS, the electrode 208 may be aligned with the opening 214 and used to transmit lesion forming energy to the tissue. A Y-adapter 222 is also provided and is connected to the tubular body 210′ in the manner described above.

Referring to FIGS. 8B, 8D and 8E, the exemplary expandable tissue cover 254 is urged between the expanded and retracted positions by a plurality of splines 256. Although the number of splines 256 may be varied to suit particular situations, the exemplary embodiment includes three splines which pass through spline lumens 258 in the tubular body 210′. The distal portions of the spines 256 exit the spline lumens 258 (as well as the tubular body 210′) by way of a set of apertures 260. The distal ends of the splines 256 re-enter the tubular body 210′ through a set of apertures 262 and are fixed in place near the distal end of the tubular member, which is preferably closed. Both sets of apertures 260/262 are located within the expandable tissue cover and the splines 256 are pre-bent so that they will assume the shape shown in FIG. 8A when urged distally. When pulled proximally though the apertures 260, the distal portions of the splines 256 will rest against the exterior of the tubular body 210′.

The tissue cover 254 is preferably sized and positioned such that it will surround the opening 214 and prevent the tissue surrounding the opening 214 from being cooled by air, blood or other biological fluids. As a result, the lesion L that is formed will be generally coextensive with the perimeter of the portion of the cover 254 that is in contact with the tissue. Additionally, although the respective configurations of the tubular body opening 214 and expandable tissue cover 254 may vary from application to application to suit particular situations, the configurations in the exemplary embodiment have been chosen to insure close contact between the tissue cover and the tissue surface TS. The exemplary opening 214 is about 0.225 inch to about 0.550 inch in length and occupies approximate 25% to 50% of the perimeter of tubular body 210′. The exemplary tissue cover 254 is about 0.457 inch to about 0.792 inch in length, i.e. it extends about 0.15 inch beyond the longitudinal edges of the opening 214, and is about 0.237 inch to about 0.354 inch wide when expanded, i.e. it extends about 0.079 inch to about 0.118 inch from the sides of the tubular body 210′. The edges 264 and 266 are also close to the opening 214. With respect to materials, the exemplary tissue cover 254 is preferably formed from a flexible, thermally insulating material such as PET. The tissue cover 254 may also be provided with physical structures, such as creases 248 (FIG. 8C), that allow the tissue cover to more easily fold up for retraction into a sheath.

Movement of the splines 256 in the exemplary tissue covering probe 252 is controlled with a handle 218′. The proximal ends of the splines 256 are respectively connected to a sliders 268 that rides along a slots 270. This allows the splines 256 to be individually controlled. [Note that one of the splines 256 has not been urged proximally in FIG. 8C.] Alternatively, each of the splines 256 could be connected to a single slider.

Turning to FIGS. 8D and 8F, the splines 256 that are associated with the apertures 260 and 262 that are aligned with the opening 214 extend generally downwardly from the tubular body 210′. This configuration creates a cavity with a small volume between the tissue cover 254 and the tissue when the tissue cover is deployed adjacent to a tissue surface. The small volume may be filled with a conductive fluid (e.g. 0.9% saline) that will increase the effective size of the electrode 208 during a lesion formation procedure to that of the area under the tissue cover 254. The splines 256 will also bend when the tissue covering probe 252 is pressed against tissue in the manner illustrated in FIG. 8C.

It should also be emphasized that, although the exemplary tissue heating and tissue covering probes illustrated in FIGS. 7A-8F are non-steerable catheter-based probes, the inventions are not so limited. To that end, the inventions also encompass steerable catheter-based, and surgical probe type tissue heating and tissue covering probes, both with and without steering capabilities.

V. Temperature Sensing and Power Control

The exemplary embodiments illustrated above preferably including one or more temperature sensors, such as thermocouples or thermistors, that are associated with the hottest portion of the electrode or other tissue heating device. For example, a thermistor 272 may be secured to the inner surface the electrode 104 as is shown in FIG. 1H. The thermisor 272 is connected to connector 274 on the handle 110 (FIG. 1A) by a signal wire 276. The connector 274 is used to connect the probe to a power supply and control device, such as a source of RF coagulation energy. Power is supplied to the electrode 104 by a signal wire 278, which is also connected to the connector 274. Suitable temperature sensors and controllers which control power to electrodes based on a sensed temperature are disclosed in U.S. Pat. Nos. 5,456,682, 5,582,609 and 5,755,715.

Although the present inventions have been described in terms of the preferred embodiments above, numerous modifications and/or additions to the above-described preferred embodiments would be readily apparent to one skilled in the art. It is intended that the scope of the present inventions extend to all such modifications and/or additions and that the scope of the present inventions is limited solely by the claims set forth below.

We claim:

1. An apparatus for heating tissue, comprising:
a support structure;
a tissue heating device carried by the support structure; and
an insulation device extending outwardly from the tissue heating device.

2. An apparatus as claimed in claim 1, wherein the support structure comprises a catheter.

3. An apparatus as claimed in claim 1, wherein the tissue heating device defines a longitudinal axis and the insulation device extends transversely to the longitudinal axis.

4. An apparatus as claimed in claim 3, wherein the tissue heating device defines longitudinal ends and the insulation device is located between the longitudinal ends of the tissue heating device.

5. An apparatus as claimed in claim 3, wherein the tissue heating device defines a perimeter and the insulation device extends around the perimeter of the electrode.

6. An apparatus as claimed in claim 1, wherein the insulation device extends longitudinally.

7. An apparatus as claimed in claim 6, wherein the tissue heating device defines a distal end and the insulation device extends around the distal end.

8. An apparatus as claimed in claim 1, wherein the tissue heating device defines a tissue contact surface on one side of the insulation device and a non-contact surface on the other side of the insulation device, the apparatus further comprising:
a layer of electrically insulating material over at least a portion of the non-contact surface.

9. An apparatus as claimed in claim 1, wherein the tissue heating device defines a slot configured to receive the insulation device.

10. An apparatus as claimed in claim 1, wherein the insulation device is retractable.

11. An apparatus as claimed in claim 1, wherein the tissue heating device comprises an electrode.

12. A tissue heating device, comprising:
an energy transmission element defining a tissue contact surface that will engage a predetermined region of tissue during a tissue heating procedure; and
an insulation device extending outwardly from the energy transmission element and positioned such that it will cover tissue surrounding the predetermined region of tissue when the energy transmission element is in contact with the predetermined region of tissue.

13. A tissue heating device as claimed in claim 12, wherein the energy transmission element comprises an electrode.

14. A tissue heating device as claimed in claim 12, wherein the energy transmission element defines a longitudinal axis and the insulation device extends transversely to the longitudinal axis.

15. A tissue heating device as claimed in claim 14, wherein the energy transmission element defines longitudinal ends and the insulation device is located between the longitudinal ends of the energy transmission element.

16. A tissue heating device as claimed in claim 14, wherein the tissue heating device defines a perimeter and the insulation device extends around the perimeter of the energy transmission element.

17. A tissue heating device as claimed in claim 12, wherein the insulation device extends longitudinally.

18. A tissue heating device as claimed in claim 17, wherein the energy transmission device defines a distal end and the insulation device extends around the distal end.

19. A tissue heating device as claimed in claim 12, wherein the energy transmission element defines a non-contact surface, the apparatus further comprising:
a layer of electrically insulating material over at least a portion of the non-contact surface.

20. A tissue heating device as claimed in claim 12, wherein the energy transmission device defines a slot configured to receive the insulation device.

21. An electrophysiology electrode, comprising:
a tissue contact surface;
an electrode cooling surface including a plurality of discontinuities; and
a substantially electrically insulating, substantially thermally conductive layer coextensive with at least a portion of the electrode cooling surface.

22. An electrophysiology electrode as claimed in claim 21, wherein the tissue contact surface is substantially smooth.

23. An electrophysiology electrode as claimed in claim 21, wherein the discontinuities comprise indentations.

24. An electrophysiology electrode as claimed in claim 23, wherein the indentations comprises longitudinally extending slots.

25. An electrophysiology electrode as claimed in claim 23, wherein the electrode cooling surface is defined by a wall having a wall thickness, the indentations defines a depth, and the depth is less than the wall thickness.

26. An electrophysiology electrode as claimed in claim 21, wherein the discontinuities comprise protuberances.

27. An electrophysiology electrode as claimed in claim 21, wherein the substantially electrically insulating, substantially thermally conductive layer defines a thermal conductivity of between approximately 200 w/m·k and approximately 500 w/m·k.

28. An electrophysiology device, comprising:
a support structure;
an electrode including a tissue contact surface and an electrode cooling surface with a plurality of discontinuities; and
a substantially electrically insulating, substantially thermally conductive layer coextensive with at least a portion of the electrode cooling surface.

29. An electrophysiology device as claimed in claim 28, wherein the support structure comprises a catheter.

30. An electrophysiology device as claimed in claim 28, wherein the tissue contact surface is substantially smooth.

31. An electrophysiology device as claimed in claim 28, wherein the discontinuities comprise indentations.

32. An electrophysiology device as claimed in claim 31, wherein the indentations comprises longitudinally extending slots.

33. An electrophysiology device as claimed in claim 28, wherein the discontinuities comprise protuberances.

34. An electrophysiology device as claimed in claim 28, wherein the electrode cooling surface is defined by a wall having a wall thickness, the indentations defines a depth, and the depth is less than the wall thickness.

35. An electrophysiology device as claimed in claim 38, wherein the substantially electrically insulating, substantially thermally conductive layer defines a thermal conductivity of between approximately 200 w/m·k and approximately 500 w/m·k.

36. A device for use with a tissue heating probe including a support structure and a tissue heating element carried by the support structure, the device comprising:
an elongate member including a side wall defining an interior region and a side wall opening; and
an expandable tissue cover carried by the elongate member and configured to cover tissue laterally adjacent to the side wall opening when in an expanded state.

37. A device as claimed in claim 36, wherein the elongate member defines a distal end and the side wall opening is proximally spaced from the elongate member distal end.

38. A device as claimed in claim 36, wherein the expandable tissue cover comprises an inflatable device.

39. A device as claimed in claim 38, wherein the elongate member includes at least one fluid lumen in communication with the inflatable device.

40. A device as claimed in claim 36, wherein the expandable tissue cover comprises a sheet and at least one spline.

41. A device as claimed in claim 40, wherein the elongate member includes at least one spline lumen.

42. A device as claimed in claim 26, wherein the expandable tissue cover comprises a thermally insulating expandable tissue cover.

43. A tissue heating apparatus, comprising:
a tissue heating probe including a support structure and a tissue heating element carried by the support structure; and
an elongate member, including a side wall defining an interior region and a side wall opening, configured to receive the tissue heating probe; and
an expandable tissue cover carried by the elongate member and configured to cover tissue laterally adjacent to the side wall opening when in an expanded state.

44. An apparatus as claimed in claim 43, wherein the support structure comprises a catheter.

45. An apparatus as claimed in claim 43, wherein the tissue heating element comprises an electrode.

46. An apparatus as claimed in claim 43, wherein the elongate member defines a distal end and the side wall opening is proximally spaced from the elongate member distal end.

47. An apparatus as claimed in claim 43, wherein the expandable tissue cover comprises an inflatable device.

48. An apparatus as claimed in claim 47, wherein the elongate member includes at least one fluid lumen in communication with the inflatable device.

49. An apparatus as claimed in claim 43, wherein the expandable tissue cover comprises a sheet and at least one spline.

50. An apparatus as claimed in claim 49, wherein the elongate member includes at least one spline lumen.

51. An apparatus as claimed in claim 43, wherein the expandable tissue cover thermally insulating expandable tissue cover.
